(19) United States
(12) Patent Application Publication
(10) Pub. No.: US 2025/0091981 A1
(43) Pub. Date: Mar. 20, 2025

(54) PROCESS FOR THE PURIFICATION OF 5-AMINOSALICYLIC ACID

(71) Applicant: CHEMI S.P.A., Cinisello Balsamo (IT)

(72) Inventors: Lorenzo DE FERRA, Patrica (IT); Fabrizio COCCHI, Patrica (IT); Mauro ANIBALDI, Patrica (IT)

(73) Assignee: CHEMI S.P.A., Cinisello Balsamo, MI (IT)

(21) Appl. No.: 18/832,045

(22) PCT Filed: Jan. 23, 2023
(86) PCT No.: PCT/IB2023/050533

§ 371 Date: Jul. 22, 2024

(30) Foreign Application Priority Data
Jan. 25, 2022 (IT) 102022000001151

(51) Int. Cl. C07C 227/42

(52) U.S. Cl. CPC C07C 227/42

(57) ABSTRACT

A process for the purification of 5-aminosalicylic acid (5-ASA) comprising the steps of: i) preparing an aqueous solution or suspension of 5-ASA, optionally added with at least another solvent; ii) adding a base until reaching a pH between 6 and 9; iii) adding an acid to the solution obtained from step ii) until reaching a pH between 3.2 and 5.2, preferably between 4.3 and 4.6, with consequent precipitation of 5-ASA; iv) separating the precipitated 5-ASA from step iii).

The object of the present invention is a process for the purification of 5-ASA. This process allows to obtain a product having a lower content of impurities and in an increased yield.

### STATE OF THE ART

5-Amino salicylic acid is known by the acronym 5-ASA or by the names mesalamine or mesalazine. It is produced in large amounts for use as an anti-inflammatory drug for the gastrointestinal tract.

Various methods for preparing 5-ASA have been proposed, among these the most frequently used are based on salicylic acid nitration and subsequent reduction of the nitro group into an amino group, or on the reaction of salicylic acid with an aromatic diazonium salt, obtained by nitrosation of the corresponding aromatic amine with sodium nitrite, to give a diazo derivative which has then to be reduced to obtain 5-ASA (Scheme 1).

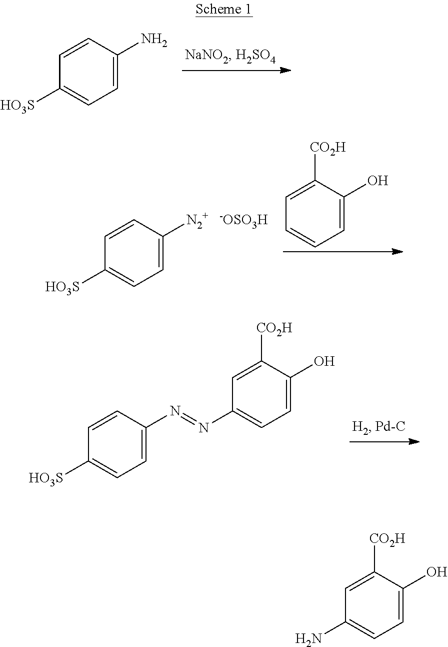

A synthetic alternative to these methods consists in the conversion of 3-nitrobenzoic acid into 5-ASA by reduction to the corresponding hydroxylamine and a Bamberger-type rearrangement of the same.

It is also possible to follow a more rarely mentioned procedure for the synthesis of 5-ASA, in which a Kolbe carboxylation of p-aminophenol is carried out using the Marasse method (Scheme 2).

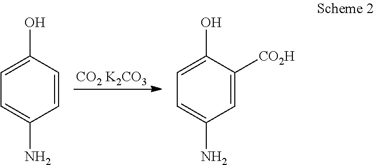

This synthetic route, despite involving the use of reactors for carrying out solid phase reactions under high pressure and temperature conditions, has the undoubted advantage of carrying out the synthesis of mesalazine with high atomic efficiency and in a single synthetic step. Furthermore, no nitrosating agents such as sodium nitrite, which carry the risk of formation of nitrosamines, are used in this process. Nitrosamines are known to be highly genotoxic substances for which health organizations have established extremely low limits that must be met in pharmaceutical products. In view of these advantages, the production of 5-ASA based on p-aminophenol carboxylation plays a role of primary importance for the production of this drug of primary utility.

Despite achieving a practically complete conversion in the carboxylation reaction, some undesired impurities are still formed also with this process. Among these, 3-carboxy-5-ASA of Formula 1 was identified:

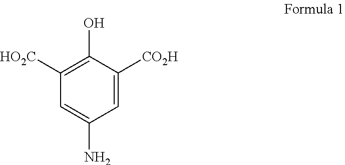 [SMILES: Nc1cc(C(=O)O)c(O)c(C(=O)O)c1]

It has been established that the presence of this dicarboxylation product in 5-ASA should be in amounts not above 0.05% by weight. This limit is not dictated by specific reasons of toxicological nature, but by the need to keep the purity profile of 5-ASA very high.

However, this limit of 0.05% cannot be met without an effective purification process since, even in the best identified conditions, the presence of 3-carboxy-5-ASA at the end of the carboxylation reactions is always higher than 0.10% by weight, and due to the tendency of this impurity to co-precipitate together with 5-ASA during its crystallization.

Until now, the purification method adopted for the removal of this dicarboxylated impurity involves 5-ASA isolation directly from the end carboxylation mixture using water as a solvent (Scheme 3). In the presence of a high concentration of potassium ions, 5-ASA precipitates from water in the form of potassium salt while the potassium salt of 3-carboxy-5-ASA is mainly found in the aqueous phase.

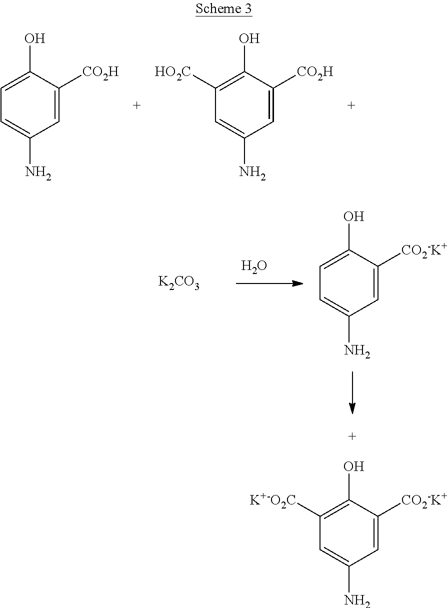

This procedure allows to achieve the desired purification of 5-ASA, but suffers from an unsatisfactory recovery yield because about 15% of 5-ASA remains in solution and is not recovered.

An improved 5-ASA production process, which achieves an increased purification yield while retaining the characteristic advantages of the procedure based on Kolbe carboxylation with high atomic efficiency and no risks of nitrosamine formation, is therefore still highly desirable.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions in the present disclosure should not be construed to represent a substantial difference over what is generally understood in the art.

The terms “approximately” and “about” used in the text refer to the range of the experimental error that is inherent in the execution of an experimental measurement.

The terms “comprising”, “having”, “including” and “containing” are to be intended as open-ended terms (i.e., meaning “comprising, but not limited to”), and are to be considered as a support also for terms such as “consist essentially of”, “consisting essentially of”, “consist of”, or “consisting of”.

The terms ““consist essentially of”, “consisting essentially of” are to be intended as semi-closed terms, meaning that no other ingredients affecting the novel features of the invention are included (optional excipients may therefore be included).

The terms “consists of”, “consisting of” are to be intended as closed terms.

### DESCRIPTION OF THE INVENTION

The present invention relates to a process for the purification of 5-ASA.

5-ASA solubility in water shows a strong pH dependence, due to the presence of ionizable functional groups in its structure.

5-ASA shows good solubility in water at a pH lower than 2, due to the presence of the primary amino group. Deprotonation of the carboxy group, on the other hand, favors 5-ASA solubility at a pH higher than 6.

In the region close to the isoelectric pH, whose value reported in the literature is equal to 3.7, 5-ASA solubility in water is minimal.

The precipitation method commonly used at the end of the synthesis for 5-ASA isolation involves the dissolution in an acid environment and the subsequent precipitation by base addition (CN111548283 and Journal of Crystal Growth 181 (1997) 403-409). On the other hand, the effects of different operating conditions for the precipitation, when this is carried out by acid addition starting from a 5-ASA aqueous solution with a pH greater than or equal to 6, are not known.

The present inventors have now surprisingly found that it is possible to obtain a better 5-ASA purification by precipitating it through acid addition starting from an aqueous solution of the same having a pH between 6 and 9. With the precipitation method according to the invention, better purifications are achieved than those deriving from the commonly adopted precipitation process consisting in the precipitation of 5-ASA from an aqueous solution of the same at acid pH by addition of bases.

This is an unexpected and surprising result since, irrespective of the addition method used, the final mixture reaches the same pH range close to the isoelectric pH of the product. The relative solubilities of 5-ASA and impurities deriving from the carboxylation reaction, first of all 3-carboxy-5-ASA, should therefore be identical and thus an identical purification effect would also be expected.

For the purposes of the present invention, different 5-ASA precipitation methods were independently tested, using as starting material the solid mixture obtained at the end of the Kolbe-Schmitt carboxylation reaction, using the Marasse variant with p-aminophenol, where the major component in the end carboxylation mixture is potassium carbonate. 5-ASA (measured as a non-salified species) is present in a percentage content of about 20% and p-aminophenol, which is the starting raw material of the synthesis, is present in the mixture for less than 0.1%. The remainder is represented by inorganic salts, mainly potassium carbonate.

This composition clearly confirms the high degree of conversion achievable thanks to carboxylation conditions used.

3-Carboxy-5-ASA, which is the species resulting from double carboxylation of p-aminophenol, is present at a level of 0.10%-0.15% with respect to 5-ASA. It is therefore above the acceptability limits of 0.05% set for this impurity.

An object of the present invention is therefore represented by a process for the purification of 5-ASA comprising the steps of:
i) preparing an aqueous solution or suspension of 5-ASA, optionally added with at least another solvent;
ii) adding a base until reaching a pH between 6 and 9;
iii) adding an acid to the solution obtained from step ii) until reaching a pH between 3.2 and 5.2, preferably between 4.3 and 4.6, with consequent precipitation of 5-ASA;
iv) separating the precipitated 5-ASA from step iii).

Other solvents which can optionally be added to the initial aqueous solution are selected from polar solvents, preferably from C₁-C₄ alcohol (methanol, ethanol or isopropanol), acetone, dimethylformamide and tetrahydrofuran, or mixtures thereof.

Once the dissolution of 5-ASA (step i) is achieved, a base (step ii) is added, preferably selected from those used in industrial practice. These include sodium hydroxide, potassium hydroxide, sodium carbonate, potassium carbonate, ammonia. More preferably, sodium hydroxide in aqueous solution.

The operation is monitored by means of a pH meter, and the base addition should be stopped when the dissolution is complete; the pH of the solution will be between pH=6 and pH=9.

In this step, the temperature should preferably be between 50° C. and 80° C., more preferably between 60° C. and 75° C.

An acidic reagent is added to this solution for 5-ASA precipitation (step iii).

For the purposes of the present invention, an acid selected from those used in industrial practice is employed. These include hydrochloric acid, sulfuric acid, phosphoric acid, acetic acid. Preferably, hydrochloric acid in aqueous solution.

The precipitation of 5-ASA should be monitored by means of a pH meter and the addition of the acid should be stopped when the pH of the mixture is between pH=3.2 and pH=5.2, preferably when the pH is between pH=4.3 and pH=4.6.

In this step, the temperature should preferably be between 50° C. and 80° C., more preferably between 60° C. and 75° C.

The precipitated product is separated from the liquid phase (step iv), preferably by means of a suitable filtering system or by centrifuge.

After drying, 5-ASA is obtained in a good yield with a residual percent content of 3-carboxy-5-ASA impurity lower than or equal to 0.05% by weight, typically lower than or equal to 0.04% by weight.

Preferably, the product is characterized in that it has a nitrosamine content lower than 1 ng/g.

In a preferred embodiment of the invention, 5-ASA is obtained from the carboxylation reaction of p-aminophenol with potassium carbonate in the presence of carbon dioxide according to Scheme 2:

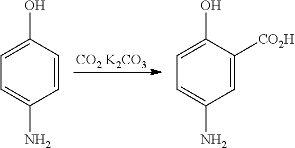

In a preferred embodiment, once 5-ASA dissolution is achieved (step i), an acid reagent is added until reaching a pH between 0.5 and 1.5, preferably equal to about 1 (step a). In this step, the temperature should preferably be between 25° C. and 70° C., more preferably between 35° C. and 50° C.

During the acid addition, abundant development of carbon dioxide is observed, and it is necessary to proceed with adequate graduality to avoid problems deriving from an excessively fast gas development. In this step, the product precipitates; the acid addition is then continued until complete redissolution.

For the purposes of the present invention, an acid selected from those used in industrial practice is employed. Among these, hydrochloric acid, sulfuric acid, phosphoric acid, and acetic acid are preferred. More preferably, hydrochloric acid in aqueous solution.

Optionally, the acid solution at the end of the redissolution may be treated with decolorizing charcoal to improve the color level.

Then, a basic reagent for 5-ASA precipitation is added to the solution (step b).

For the purposes of the present invention, a base selected from those in use in industrial practice is employed. Among these, sodium hydroxide, potassium hydroxide, sodium carbonate, potassium carbonate and ammonia are preferred. More preferably, sodium hydroxide in aqueous solution.

The precipitation of 5-ASA must be monitored by means of a pH meter, the addition of the base to the acid should be stopped when the pH of the mixture is between pH=3.0 and pH=4.0, preferably between 3.5 and 3.7, more preferably when a pH of about 3.5 is reached.

In this step, the temperature should preferably be between 50° C. and 80° C., more preferably between 60° C. and 75° C.

Optionally, the precipitated product is separated from the liquid phase (step c), preferably by means of a suitable filtering system or centrifuge. The solid is optionally dried.

Optionally, an aqueous solution or suspension of the 5-ASA precipitated from step c), optionally added with at least one other solvent, is prepared before carrying out step ii).

Alternatively, product isolation (step c) is not performed after the precipitation step with bases (step b) and the addition of the basic reagent is continued until redissolution of 5-ASA (step ii).

Also in this alternative embodiment, the operation is monitored by means of a pH meter and the addition of the base should be stopped when the dissolution is complete, the pH of the solution will be between pH=6 and pH=9.

Also in this embodiment, an acid reagent is then added to the solution for precipitating 5-ASA (step iii) until reaching a pH between pH=3.2 and pH=5.2; preferably when the pH is between pH=4.3 and pH=4.6.

This preferred embodiment demonstrates the versatility of the invention which allows to obtain a good purification level through the precipitation of the product also from an environment with a high ionic strength due to the presence of the potassium carbonate salts used in the carboxylation reaction, and in the absence of intermediate isolation.

For comparative purposes, a sequence of operations similar to that according to the invention was carried out, without performing the precipitation with an acid reagent, but replacing it with a second precipitation using a basic reagent and adjusting the final pH in the range between pH=4.3 and pH=4.6.

The following table shows the data relating to the various preparations described in the Examples.

TABLE 1

| | | 3-Carboxy-5-ASA percent | 5-ASA percent | |
|---|---|---|---|---|
| | | content in the isolated | content in the | |
| Example | Description | product | mother liquor | Yield |
| 1 | Carboxylation | 0.10% | Not present in | NA |
| | reaction | | mother liquor | |
| 2 | Precipitation with a | 0.09% | 2.4 g/L | 97.3% |
| | base according to | | | |
| | the prior art | | | |
| 3 | Precipitation with | 0.02% | 2.5 g/L | 94.9% |
| | an acid according | | | |
| | to the invention | | | |
| 4 | Carboxylation | 0.12% | Not present in | NA |
| | reaction | | mother liquor | |
| 5 | Precipitation with a | 0.12% | 1.5 g/L | 93.9% |
| | base according to | | | |
| | the prior art | | | |
| 6 | Precipitation with | 0.04% | 2.1 g/L | 97.7% |
| | an acid according | | | |
| | to the invention | | | |
| Comp. | Precipitation with a | 0.10% | 1.8 g/L | 97.2% |
| Ex. 1 | base according to | | | |
| | the prior art | | | |

NA = not available

Table 1 clearly shows the high efficiency of the purification carried out by precipitation with acid, as described in Examples 3 and 6 according to the present invention. The amount of 5-ASA remaining in the precipitation mother liquor is low, a further advantage of this method is therefore the obtainment of good isolation yields, typically close to 96%, therefore well higher than the 85% deriving from the purification procedure based on the isolation of 5-ASA as a potassium salt.

Surprisingly, the purification effect found by applying the procedure object of the present invention cannot be attributed solely to the choice of the pH at which the isolation of 5-ASA is carried out.

The comparison of the results of Example 6 with those of Comparative Example 1 demonstrates that:

Example 6 describes the purification performed according to the invention, with precipitation of the product by adding acid to the basic solution obtaining a final 3-carboxy-5-ASA impurity value equal to 0.04%.

In Comparative Example 1, carried out starting from the same 5-ASA preparation, the precipitation is continued until the same pH level is obtained by addition of a base to an acid solution (and not vice versa): the final level of 3-carboxy-5-ASA impurity is 0.10% and above the limit of 0.05% by weight.

Another advantage deriving from the accomplishment of this invention is the absence of nitrosamines in the 5-ASA obtained. As known, nitrosamines are highly genotoxic impurities and the regulatory authorities, such as EMA and FDA, have set very stringent limits for this class of compounds, specifying that due to their presence, the risk analysis must be carried out for the class of nitrosamines as a whole. It is therefore particularly useful to have a method which allows to measure the level of the nitrosamino group, —NNO, independently of the specific molecule to which it is bound.

The luminescence-based method described for example in the article by Beretta et al., Journal of Pharmaceutical and Biomedical Analysis 49 (2009) 1179-1184, incorporated herein by reference, appears to be perfectly adequate for this need.

By means of this analysis method, the amount of nitrosamino group in a sample of 5-ASA (tablets for pharmaceutical human use commercially available in the USA) and in the 5-ASA obtained in Example 6 according to the invention were measured.

The results obtained are shown in Table 2.

TABLE 2

| Sample | −NNO group (ng/g) |
|---|---|
| 5-ASA tablets from USA market | 2.0 |
| 5-ASA from Example 6 | Below the Detection Limit |

Note:
Detection Limit = 1 ng/g

These data confirm the absence of nitrosamines in the product obtained according to the process of the present invention.

The presence of the nitrosamino group was instead identified in the 5-ASA tablet from the US market. It is possible to evaluate how the level found compares with the regulations that have recently been introduced on the subject.

The tablet weighs 1.44 g and has a 5-ASA content of 1.2 g.

Therefore, calculating the ratio between the total amount of the NNO group and the active ingredient, the results is that 2.4 ng of —NNO group are contained in each gram of 5-ASA.

It is possible to evaluate the risk related to the presence of a compound containing the nitrosamino group, but whose structure is not defined, by equating its toxicity to that of nitrosamine for which the highest level of genotoxicity has been found. This is the approach that may be adopted for best protecting the patient.

N,N-diethyl-nitrosamine (NDEA), whose molecular weight is 102 Dalton, is the species with the highest levels of genotoxicity determined.

Comparing the amount of —NNO group (whose molecular weight is 44 Dalton) to NDEA, its amount in the 5-ASA contained in the analyzed tablet would correspond to 5.57 ng/g of NDEA.

The maximum daily intake limit for NDEA established in the guidelines is 26.5 ng; considering the dosage of 5-ASA which provides for a maximum daily intake of 5ASA of 4.8 g, a maximum limit for NDEA in 5-ASA equal to 5.52 ng/g is calculated.

The data in Table 2 therefore show that there are commercially available finished products based on 5-ASA in which species containing the nitrosamino group have been detected in amounts very close to the limits established for the most genotoxic substances in the category. Therefore, the presence of nitrosamines in 5-ASA represents a real issue that the implementation of the present invention unexpectedly allows to solve.

### EXAMPLES

The following examples are to be intended as specific embodiments of the invention only and are not to be construed as limiting its interpretation and field of application.

### Example 1. Carboxylation Reaction of p-aminophenol for the Preparation of 5-ASA

1025 kg of potassium carbonate and 225 kg of p-aminophenol are charged into a special reactor dedicated to solid phase reactions equipped with a mechanical stirrer and built to withstand high pressure and temperature operating conditions. The system is pressurized with carbon dioxide up to 30 Bar, the temperature is brought to 180° C. and the mixture is kept under stirring for 4 hours. The solid mixture is cooled, the reactor is depressurized and the solid obtained is discharged and analyzed by HPLC. 5-ASA titer in this solid is 20.1% while the content of 3-carboxy-5-ASA impurity in this preparation was found to be 0.10% by weight with respect to 5-ASA.

A Restek Pinnacle II C8 150×4.6 mm 5 μm column is used for HPLC analyses. For the preparation of the mobile phase, 1.39 g of KH₂PO₄ and 2.24 g of sodium octanesulphonate are dissolved in 1000 ml of water. Then, a mixture of this solution with methanol and acetonitrile is prepared in the proportions 1000:90:35 (by volume), respectively. The analysis is carried out in isocratic mode at 1 mL/minute using a UV detector at 220 nm for the detection.

### Example 2. Precipitation with a Base According to the Prior Art

180 ml of water are added to 120 g of the solid mixture obtained by the carboxylation reaction in Example 1; ˜3.5M Hydrochloric Acid is added to the suspension until reaching pH=1, thus obtaining complete dissolution. This addition should be performed gradually to limit the formation of large amounts of foam. During the addition, the temperature is gradually raised until reaching about 45° C. The solution is treated with decolorizing charcoal at 80° C. The solution is brought to 65-70° C. and a 30% aqueous solution of sodium hydroxide is added until reaching pH=3.5, thus obtaining abundant precipitation.

The mixture is gradually cooled down to 25° C. and the suspension is then kept under stirring for 15 minutes.

It is filtered on Büchner, and 25.3 g of 5-ASA are obtained after drying under vacuum at ˜50° C.

The HPLC analysis shows a titer of 92.8% for this product, while the content of 3-carboxy-5-ASA impurity is 0.09%.

The precipitation mother liquors are also analyzed by HPLC, finding a concentration of 5-ASA equal to 2.4 g/L.

### Example 3. Precipitation with an Acid According to the Invention

120 mL of water are added to 24 g of the product obtained in Example 2.

A 0.7M sodium hydroxide solution is then added until pH ˜7.4 is reached. It is heated to 70° C. and 5M hydrochloric acid is added to the solution. By maintaining temperature and stirring, the addition of acid is continued until the pH of the mixture reaches the value of pH=4.5. Precipitation is observed during this step. The addition of acid is stopped and the temperature is brought to 20-25° C. while maintaining stirring.

It is filtered on Büchner obtaining 22.3 g of 5-ASA after drying under vacuum at ˜50° C.

The HPLC analysis shows a tier of 97.8% for this product and the 3-carboxy-5-ASA impurity content is 0.02%.

The precipitation mother liquors are also analyzed by HPLC, finding a concentration of 5-ASA equal to 2.5 g/L.

### Example 4. Carboxylation Reaction of p-aminophenol for the Preparation of 5-ASA

The carboxylation reaction of p-aminophenol was carried out under the same conditions reported for Example 1. The 5-ASA assay in the product obtained was 23.1%, while the 3-carboxy-5-ASA impurity content in this preparation was found to be 0.12% by weight with respect to 5-ASA.

### Example 5. Precipitation with Base According to the Prior Art

The precipitation of 5-ASA was carried out under the same conditions reported for Example 2, using in this example the carboxylation mixture obtained from Example 4.

The HPLC analysis shows a titer of 97.2% for this product while the 3-carboxy-5-ASA impurity content is 0.12%.

The precipitation mother liquors are also analyzed by HPLC, finding a concentration of 5-ASA equal to 1.5 g/L.

### Example 6. Precipitation with Acid According to the Invention

5-ASA precipitation was carried out under the same conditions reported for Example 3, using in this example the product obtained from Example 5.

HPLC analysis shows for this product a 3-carboxy-5-ASA impurity content of 0.04%.

The precipitation mother liquors are also analyzed by HPLC, finding a concentration of 5-ASA equal to 2.1 g/L.

### Comparative Example 1. Precipitation with a Base According to the Prior Art

In this example, for comparative purposes, the same product used in the precipitation described in Example 5 was reprecipitated following a procedure which does not incorporate the teachings of the present invention but nonetheless provides for 5-ASA isolation in the same pH range.

200 ml of water are added to 20 g of 5-ASA obtained from Example 5; the suspension is heated to 60° C. and ˜10M Hydrochloric Acid is added, obtaining complete dissolution. The solution is treated with decolorizing charcoal at 80° C. The solution is brought to about 65° C. and 5M sodium hydroxide is added until reaching pH=4.4, obtaining abundant precipitation.

The temperature and stirring were maintained for one hour.

The mixture is gradually cooled down to 25° C. and the suspension is then kept under stirring for 15 minutes.

It is filtered on Büchner obtaining 18.9 g of 5-ASA after drying under vacuum at ˜50° C.

The HPLC analysis shows for this product a 3-carboxy-5-ASA impurity content of 0.10%.

The precipitation mother liquors are also analyzed by HPLC, finding a concentration of 5-ASA equal to 1.8 g/L.

1. A process for the purification of 5-aminosalicylic acid (5-ASA) comprising the steps of:
i) preparing an aqueous solution or suspension of 5-ASA, optionally added with at least another solvent;
ii) adding a base until reaching a pH between 6 and 9;
iii) adding an acid to the solution obtained from step ii) until reaching a pH between 3.2 and 5.2, preferably between 4.3 and 4.6, with consequent precipitation of 5-ASA;
iv) separating the precipitated 5-ASA from step iii).

2. The process according to claim 1, characterized in that said at least another solvent is a polar solvent, preferably selected from C₁-C₄ alcohol, acetone, dimethylformamide, tetrahydrofuran, or mixtures thereof.

3. The process according to claim 2, characterized in that said C₁-C₄ alcohol is methanol, ethanol, or isopropanol.

4. The process according to claim 1, characterized in that 5-ASA is obtained by carboxylation of p-aminophenol with potassium carbonate in the presence of carbon dioxide.

5. The process according to claim 1, characterized in that said steps ii) and iii) are carried out at a temperature between 50° C. and 80° C., preferably between 60° C. and 75° C.

6. The process according to claim 1, characterized in that the 5-ASA obtained from step iv) has a 3-carboxy-5-ASA content lower than or equal to 0.05% by weight, preferably lower than or equal to 0.04% by weight.

7. The process according to claim 1, characterized in that the nitrosamine content in the 5-ASA obtained from step iv) is lower than 1 ng/g.

8. The process according to claim 1, characterized in that the following steps are carried out upstream of step ii):
a) adding an acid to the solution or suspension obtained from step i) until reaching a pH between 0.5 and 1.5, preferably equal to about 1;
b) adding a base to the solution obtained from step a) until reaching a pH between 3.0 and 4.0, preferably between 3.5 and 3.7, more preferably equal to about 3.5, with consequent precipitation of 5-ASA;
c) optionally, separating the precipitated 5-ASA from step b) and preparing an aqueous solution or suspension of the precipitated 5-ASA, optionally added with at least another solvent.

9. The process according to claim 8, characterized in that said step a) is carried out at a temperature between 25° C. and 70° C., preferably between 35° C. and 50° C.

10. The process according to claim 8, characterized in that said step b) is carried out at a temperature between 50° C. and 80° C., preferably between 60° C. and 75° C.

11. The process according to claim 1, characterized in that said acid is selected from hydrochloric acid, sulfuric acid, phosphoric acid, acetic acid, or mixtures thereof.

12. The process according to claim 1, characterized in that said base is selected from sodium hydroxide, potassium hydroxide, sodium carbonate, potassium carbonate, ammonia, or mixtures thereof.
